(19) United States
(12) Patent Application Publication
(10) Pub. No.: US 2011/0293573 A1
(43) Pub. Date: Dec. 1, 2011

(54) METHOD AND APPARATUS FOR CULTURING TISSUE

(75) Inventors: YI-YOU HUANG, TAIPEI (TW); CHIN-HSIUNG HSIEH, TAIPEI (TW); JO-LING WANG, TAIPEI (TW)

(73) Assignee: NATIONAL TAIWAN UNIVERSITY, TAIPEI (TW)

(21) Appl. No.: 12/971,709

(22) Filed: Dec. 17, 2010

(30) Foreign Application Priority Data
May 27, 2010 (TW) 099117089

(51) Int. Cl. A61K 35/36; A61P 17/14; C12M 3/00; C40B 50/06; C12N 5/071

(52) U.S. Cl. 424/93.7; 506/26; 435/401; 435/289.1

(57) ABSTRACT

A method for culturing microtissue is provided. The method includes steps of: (a) forming a pattern microarray on a hydrophobic film; (b) adhering the hydrophobic film to a carrier; (c) disposing a plurality of cells on the hydrophobic film for culturing depending on the pattern microarray, and forming a plurality of hair follicle microtissues.

### CROSS-REFERENCE TO RELATED APPLICATION AND CLAIM OF PRIORITY

The application claims the benefit of Taiwan Patent Application No. 099117089, filed on May 27, 2010, in the Taiwan Patent and Trademark Office, the disclosures of which are incorporated herein in their entirety by reference.

### FIELD OF THE INVENTION

The present invention relates to a method for cells cultivation, in particular to a method for cells cultivation for hair follicle microtissues.

### BACKGROUND OF THE INVENTION

Tissue engineering is a technology used for cultivating and transplanting cells which can be applied in repair of tissue and organ. Tissue engineering is already widely applied to deal with each kind of medical issue with the prosperity of biotechnology. For example, doctors can treat patients with tissue transplantation by using derma and cartilage which are artificially cultivated.

Take an example for treating alopecia with embedding hair. If the hair quantity on the occipital of hindbrain is sufficient, the hair follicles of the occipital scalp can be transplanted to the baldheaded area. However this method is only an autologous transplantation of hair follicles to redistribute the position of hair follicles, it still can not produce new ones. For nearly all baldheaded patients, this method is apparently not applicable.

Furthermore, a large amount of cells must be provided in the above method, and the generating efficiency of new hair follicles is unclear, and in additional, the direction of new born hair can not be effectively controlled for the time being. Further, since the dermal papilla cells of hair follicles are typically aggregated as a tight regiment in a living creature, the amount of the transplanted cells should be large and tightly disposed to ensure having enough induced signal produced from the dermal papilla cells. A loosen distributed density of cells can not produce new hair follicles. Furthermore, the thickness of hair is in association to the size of dermal papilla cells, the amount of implanted cells must be controlled to produce the same thickness of hair for same size of new born hair follicles. There are numerous factors which can effect the solving of transplantation issues, and this result increases its difficulty.

From above mentioned, a new cultivation method and apparatus for microtissues are needed urgently. Thus, based on the drawbacks of prior art, the inventor gave the utmost attention and finally invented the cultivation method for microtissues with experiment and research. Based on the spirit to work with perseverance, the problem of prior art was solved. The particular design in the present invention not only solves the problems described above, but also is easy to be implemented. Thus, the invention has the utility for the industry.

### SUMMARY OF THE INVENTION

The original concept is to FIG. out a method for tissue cultivation which can cultivate hair follicle derma and epidermis cells to develop toward normal hair follicle style so as to ensure the transplantation efficiency, and can rapidly produce a large amount of microtissue array which can induce the growth of hair. In addition, the hair has appropriate arrangement direction after being transplanted to desired area.

According to above thought, a method for tissue cultivation, which includes steps of: (a) forming a patterned microarray on a hydrophobic membrane; (b) attaching the hydrophobic membrane to a carrier; (c) disposing plural cells on the hydrophobic membrane for the cultivation of microtissue; and (d) causing the plural cells to form plural hair follicle microtissues on the carrier according to the patterned microarray.

Preferably, the present invention which addresses a method for tissue cultivation further includes a step of forming a transplantation area on a dermal surface and disposing the hair follicle microtissue on the transplantation area.

Preferably, the present invention which addresses a method for tissue cultivation further includes a step of attaching the hair follicle microtissue to a substrate and cohering the substrate on the transplantation area to transplant the hair follicle microtissue on the transplantation area.

Preferably, the present invention which addresses a method for tissue cultivation, wherein the patterned microarray includes plural holes, and each of the plural holes has a diameter ranged from 200 μm to 800 μm.

Preferably, the present invention which addresses a method for tissue cultivation, wherein the plural cells include at least one being selected from a group consisting of an embryonic stem cell, a mesenchymal stem cell, a dermal papilla cell, a hair follicle stem cell, a hematopoietic stem cell, a dermal cell and an epidermis cell.

According to above thought, a method for tissue cultivation includes the steps of: (a) providing a membrane; (b) forming a hole on the membrane; (c) attaching said membrane to a carrier; (d) causing the hole to form a microwell with the carrier; (e) disposing plural cells on the membrane; and (f) cultivating the plural cells to form a microtissue in the microwell.

According to above thought, an apparatus for microtissue cultivation which includes a carrier; and a membrane disposed on the carrier and having a hole where plural cells are cultivated to form a microtissue.

Preferably, the present invention which addresses an apparatus for tissue cultivation which includes a substrate to cultivate the microtissue and to be cohered on a transplantation area to transplant the microtissue.

Preferably, the present invention which addresses an apparatus for tissue cultivation, wherein the membrane is a hydrophobic membrane and the material of the membrane includes at least one being selected from a group consisting of a siloxane, an alkene and a polycarbonate, and the carrier includes at least one being selected from a group consisting of a glass, a ceramics, a polystyrene, a silicon wafer, a gelatin and a metal.

According to above thought, an apparatus for microtissue cultivation which includes a membrane having a microwell for cultivating plural cells into a microtissue therein.

The present invention may best be understood through the following descriptions with reference to the accompanying drawings, in which:

### BRIEF DESCRIPTION OF THE DRAWINGS

FIG. 1 is a diagram showing a membrane which is segmented by cutting machine according to an embodiment of the present invention;

FIG. 2 is a diagram showing a membrane after cutting according to an embodiment of the present invention;

FIG. 3 is a diagram showing membranes which are attached to a carrier according to an embodiment of the present invention; and

FIG. 4 is a diagram showing cultivated cells which are centralized in microwells structure according to an embodiment of the present invention.

FIG. 5 is an optical image showing cultivated microtissues in different sizes of holes according to an embodiment of the present invention.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purposes of illustration and description only; it is not intended to be exhaustive or to be limited to the precise form disclosed.

In the embodiment of this invention, a method for microtissues cultivation, which includes: (a) forming a patterned microarray on a hydrophobic membrane; (b) attaching the hydrophobic membrane to a carrier; (c) disposing plural cells on the hydrophobic membrane for the cultivation of microtissue; and (d) causing the plural cells to form plural hair follicle microtissues on the carrier according to the patterned microarray.

The membranes utilized in this embodiment of this invention are hydrophobic and may be, but not limited to, made of a siloxane, an alkene or a polycarbonate. For example, polydimethylsiloxane (PDMS) of siloxane polymer may be used for the membrane due to its good oxygen infiltration, low surface energy and hydrophobic surface.

For manufacturing the hydrophobic membrane, first of all, PDMS and crosslinker can be mixed with a ratio of 10 to 1 in weight to form solution and then eliminate bubbles within the solution by using a vacuum ball. A suitable amount of solution is obtained and is smeared rotationally on a surface of base material (such as glass) equally. The PDMS will be formed as a membrane of PDMS by a crosslinking reaction after a few days.

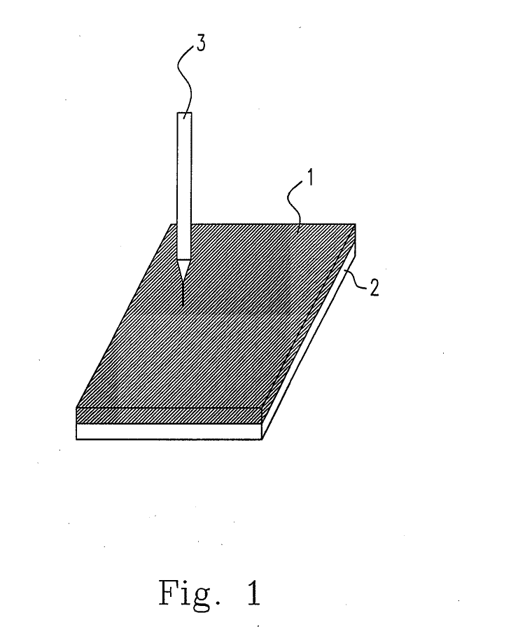

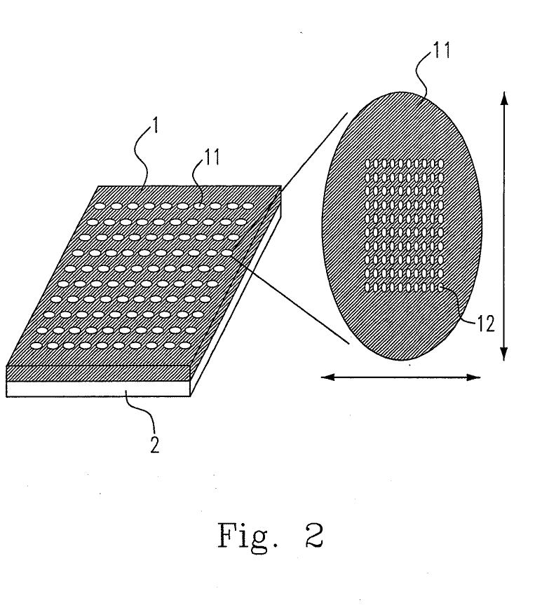

Next, the base material 2 covered with PDMS membrane 1 is disposed under the cutting machine 3 as shown in FIG. 1. FIG. 1 shows that the membrane 1 is segmented by the cutting machine 3 according to an embodiment of the present invention. In FIG. 1, the cutting machine 3 is segmenting the membrane 1 to form particular pattern arrays which has plural holes 12 on that membrane 1 as shown in FIG. 2. FIG. 2 shows a diagram of a membrane 11 after cutting according to the embodiment of the present invention. In this embodiment the diameter of the membrane 11 is around 15 mm, and the diameters of those holes 12 on the membrane 11 are around 200 μm to 800 μm. For example, the total amount of holes 12 may be hundreds to thousands. Laser sintering machine may be used for the cutting machine 3, but not limited thereto, and a microarray spotter can be selected to form plural holes on the membrane. The shape of patterned arrays is not limited to a particular shape. As for forming particular patterned arrays on the membrane 1, the patterned arrays can be depicted through AutoCAD on computer and then patterned array material can be inputted to the cutting machine 3 such as the laser sintering machine. The particular patterned arrays to fit in with necessity can be burned on the membrane 1 after setting appropriate parameters. The plural membranes 11 with particular patterned arrays should be cleaned by alcohol many times after the cutting is finished, and then, the membrane can be stored in alcohol for preparation after confirming there being not extra burned object and dust existing.

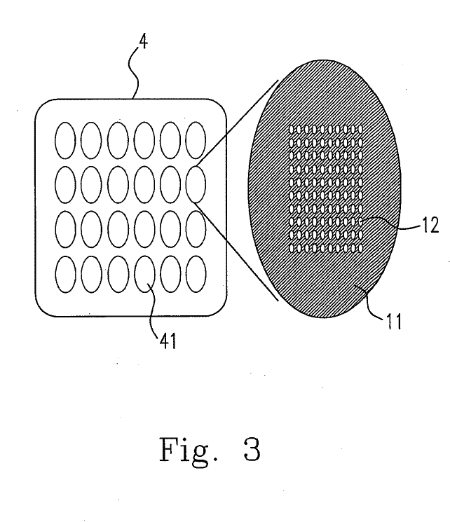

A few of alcohol can be applied to make those membranes to be attached to a carrier 4 after the preparation of the PDMS membrane as shown in FIG. 3. FIG. 3 shows a diagram of membranes attached to the carrier 4 according to an embodiment of the present invention. The carrier 4 adopted in FIG. 3 may be a fillister 41 with 24 corning, but not limited thereto, and other materials such as glass, ceramics, polystyrene, poly N-isopropylacrylamide, silicon wafer, gelatin and metal can be chosen to form the carrier 4. The shape of those membranes 11 can be matched up with and attached to the cell cultivation fillister 41 if the carrier 4 is the commercially available 24 corning. Those membranes 11 are disposed on the carrier 4, and the holes 12 of the membranes 11 form a microwell structure with carrier 4. Those are cleaned by phosphate-buffered isotonic saline (PBS) and join 0.5 ml DMEM (Dulbecco's Modified Eagle Medium, GIBCO) culture medium which contains 10% of fetal bovine serum (FBS), then put into the cell cultivation box for half an hour. After the extra air of DMEM cultivation solution attached to membranes 11, bubbles are removed by a pipet to continue to process cell cultivation.

The cultivated cells in this embodiment of the present invention may be an embryonic stem cell, a mesenchymal stem cell, a dermal papilla cell, a hair follicle stem cell, a hematopoietic stem cell, a dermal cell, an epidermis cell or a combination thereof. The mesenchymal stem cells which are located within bone marrow are belonged to one kind of adult stem cells, and they are easily separated and cultivated with fast proliferation in vitro. Those stem cells can be differentiated to particular function of cells under stimulation of appropriate growth factors and are suitable due to possessing differentiation ability of similar a dermal papilla cell and a connective tissue sheath cell.

The dermal papilla cell are tight cell mass within a human body, and the implanted microtissues should be large in quantity and disposed close together to ensure enough induced signal produced from dermal papilla cells, in the other side, the cells will not produce new hair follicles if the microtissues are excessively loosen. Furthermore, since the thickness of hair is in relation to the size of dermal papilla cells, the amount of implanted cells must be controlled to produce same thickness of hair for same size of new born hair follicles. Thus, in the embodiment of this invention, the holes on the membrane used for cultivating cells may have diameters ranged from 200 μm to 800 μm and preferably from 200 μm to 400 μm.

Afterwards, a suitable amount of cultivated cells are extracted, and the dermal papilla cells with cell quantity counted by hemocytometer counter are put into a centrifugal machine with a rotation speed of 1,000 rpm for ten minutes. Those cells are attached into the above mentioned carrier 4 with particular patterned arrays of the membrane segments, then a culture medium is joined, and the cells may be cultivated, for example, in cells cultivation box (REVCO) with 5% CO₂ in 37 centigrade. The carrier 4 is drawn out every half an hour from the cells cultivation box and is shaken 3 to 4 times to bring the cells equally distributed on the membranes.

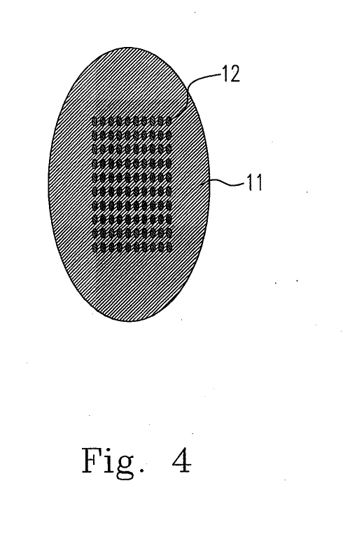

FIG. 4 is a diagram of the cultivated cells centralized in the plural microwell structure which is formed with the plural holes 12 and the carrier 4 of membrane 11. Since PDMS is a high hydrophobic material, most cells do not attach to PDMS membranes, and the cells attach to culture medium of the carrier 4 by utilizing the nature of cells not being attached to PDMS, thus, the dermal papilla cells will be quite restrained and heaped in microwells. A large amount of cells can grow in the microwell 12 with narrow space, therefore, an objective of a local high density of cells quantity and area is achieved, and the microtissue can be formed for transplantation.

Although the above mentioned embodiment of this invention is to dispose membrane on a carrier to cultivate microtissue, the membrane can directly form microwells as well, and the bottom of microwell come into being suitable environment for cells to be attached and grow such as disposing the attachable culture medium in the bottom of microwell and then a microtissue can be formed by disposing and cultivating cells in the structure of microwell. This is to say, the membrane itself can contain the effect of carrier and this characteristic is also in the scope of this invention.

The formation of dermal papilla microtissue is observed.

In order to confirm the efficacy of the embodiment of this invention, the formation result of microtissue in the embodiment is observed below.

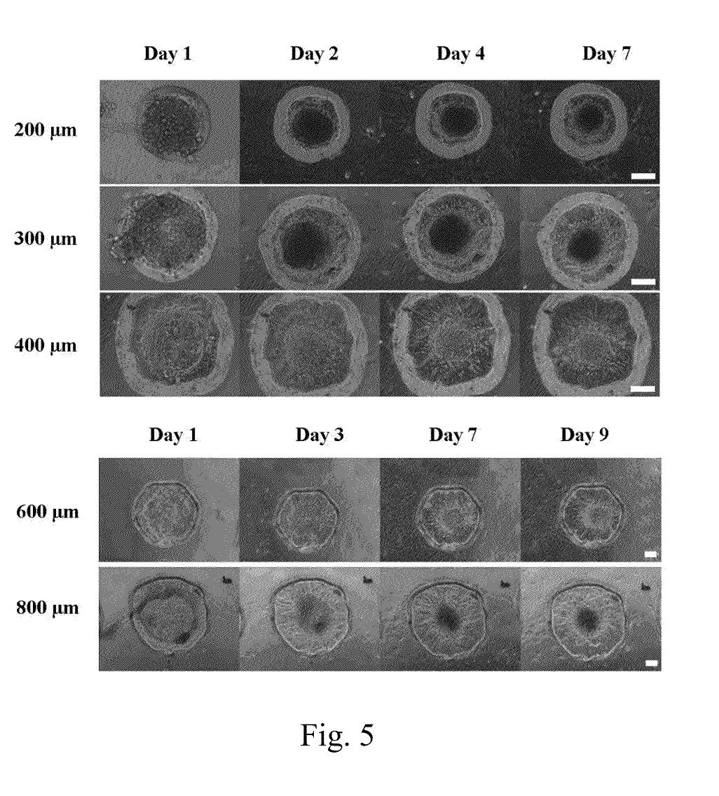

Referring to FIG. 5, it shows an optical image of cultivated microtissues in different sizes of holes according to the embodiment of the present invention. A 4×10⁵ cells are put in the microwell structure which is formed of individual holes and the carrier of membrane, and the cultivated dermal papilla will have the status of piling up in holes the next day in a size of 200 μm, 300 μm of microarray, but a similar cell mass of microtissue is not produced. A microtissue with a uniform ball shape which can be viewed with borders has the phenomenon of centralization apparently, and this microtissue will get together more tightly to cause a shrinkage in size after the third or fourth day, then the cells on borders will spread out toward the bottom of culture medium and this will lead to disappearance of borders of the original microtissue, and the status is maintained afterwards. And then the cultivation situations of dermal papilla cells in a hole with a bigger diameter and a hole with a small diameter are not quite the same. Initially there is not phenomenon of full out with overcrowded cells, but the upper layer of cells are gradually stacked after the bottom layer being posted level, after that these stacked cells will congregate toward one point. The formed microtissue is not like those in microholes with a small diameter to have apparent ball shape, but a stacked situation of a similar pillow shape is observed in certain range.

Besides, according to the experimental result of the embodiment in this invention revealed, wherein the 5×10⁴ cells given to a 24 corning with measured area in 1.9 cm² will not form cell mass no matter what are the size of microholes. The cell mass can be formed in smaller size of microholes such as 200 μm, 300 μm and 400 μm when the cells being increased to above 1×10⁵ in quantity, however the most portion of cultivated cells is with the situation of posted level to the culture medium, only small amount of cells will be stacked to form microtissue, hence the cultivated cell quantity will effect the formation of microtissue.

The microtissues which are similar to the dermal papilla taken from the beard of an ordinary mouse can be obtained efficiently within three days to five days by adoption of high cell density and a small size of microholes which are included in the patterned array with plural microwells structure. Since the size of dermal papilla from the beard of the ordinary mouse is ranged from 100 μm to 200 μm, the microtissues obtained in the patterned array with a hole size ranged from 200 μm to 400 μm are located within this range. Although there is formation of the dermal papilla microtissue under the cell density of 5.26×10⁴/cm², the inventor discovered that the most large quantity and integrated model of microtissue was formed in cell density of 2.1×10⁵/cm². For mass production, although the quantity of membrane holes is 100/1.9 cm² in the experiment, a 300 to 500 holes according to the hole sizes can be obtained on size of 1.9 cm², and the smaller size of membrane holes can have the more large number of holes and obtain more usable microtissue efficiently.

In order to prove the effect of microtissue cultivation, a activity staining is applied to dermal papilla microtissue for different cultivation periods by utilizing Live/Dead Viability Kit, the experiment of activity staining for cell microtissues having cultivated for five days discovered that the microtissue show a large amount of green fluorescence and not show nearly any red fluorescence, and the appearance represents the cell is reactivated in these microtissues and only a few of cells are causing death. However, the experiment with microtissue cultivated with twelve days discovered red fluorescence in the middle portion, and the appearance represents internal portion of the microtissue cells being caused death, and after the cultivation which is experienced with many days, the cell in the internal of microtissue could not be obtained enough nutrition and the situation of causing death of internal cells is foreseeable.

For observation of whether these dermal papilla microtissues possess the induced ability to hair follicle revival, a anti-aSMA and a anti-NCAM antibodies with cell marks of a-smooth muscle actin which is shortened to a-SMA and NCAM to proceed the experiment of immune fluorescence dyeing for dermal papilla microtissue. In part of the experiment, the microtissue mass can smoothly dyed with these cell marks by adopting cultivation of three days, cultivation of five days and cultivation of seven days, however, wherein the dyeing experiment is performed after extended twelve days, the fluorescence strength in these cell mass is very weak. Many cells in microtissue after multiple days of cultivation are causing death, hence the stored a-SMA and NCAM is a few and the fluorescence strength is very weak. According the above result, the PDMS membrane with plural holes of patterned array can limit the ambit of cell growth and the forming dermal papilla microtissue actually contain the growth activity and possess the particular cell marks, however a cultivation in long period of time is not possible, because portion of cells will be causing death and loosing the capability to induce revival of hail follicle.

The transplantation of microtissues.

Owing to the short working range of induced signal generated from dermal papilla, the position of transplantation in vitro should be very close to epidermis of the dermal cell to ensure effective propagation of induced signal, or the dermal papilla cell and epidermis cells should be implanted to a deeper position under skin and direct the induced signal to propagate to epidermis cells. The microtissue which is cultivated with the method provided by the embodiment of this invention can be transplanted in vitro with transplantation methods of forming a transplantation area such as a wound on a dermal surface and disposing the hair follicle microtissue on the transplantation area, or attaching the cultivated microtissue to a substrate which can be a thing such as artificial skin and cohering the substrate on the transplantation area to transplant the hair follicle microtissue on the transplantation area. There are of course other transplanting methods, and any transplanting method to adopt cultivated microtissue of this invention is belonged the application area of this invention.

In summary the embodiment of above mentioned invention, the patterned array microtissue which is cultivated from the method of an embodiment of this invention can be developed toward normal hair follicles style and be guaranteed the transplantation efficiency, and can rapidly manufacture a large amount of microtissue array which can induce the growth of hair. In addition, the hair has appropriate arrangement direction after transplanting to desired area.

While the invention has been described in terms of what are presently considered to be the most practical and preferred embodiments, it is to be understood that the invention need not be limited to the disclosed embodiment. On the contrary, it is intended to cover various modifications and similar arrangements included within the spirit and scope of the appended claims, which are to be accorded with the broadest interpretation so as to encompass all such modifications and similar structures. Therefore, the above description and illustration should not be taken as limiting the scope of the present invention which is defined by the appended claims.

What is claimed is:

1. A method for a cultivation of microtissue, comprising the steps of:
(a) forming a patterned microarray on a hydrophobic membrane;
(b) attaching the hydrophobic membrane to a carrier;
(c) disposing plural cells on the hydrophobic membrane for the cultivation of microtissue; and
(d) causing the plural cells to form plural hair follicle microtissues on the carrier according to the patterned microarray.

2. The method according to claim 1, further comprising forming a transplantation area on a dermal surface and disposing the hair follicle microtissue on the transplantation area.

3. The method according to claim 2, further comprising attaching the hair follicle microtissue to a substrate and cohering the substrate on the transplantation area to transplant the hair follicle microtissue on the transplantation area.

4. The method according to claim 1, wherein the patterned microarray comprises plural holes and each of the plural holes has a diameter ranged from 200 μm to 800 μm.

5. The method according to claim 1, wherein the plural cells comprise at least one being selected from a group consisting of an embryonic stem cell, a mesenchymal stem cell, a dermal papilla cell, a hair follicle stem cell, a hematopoietic stem cell, a dermal cell and an epidermis cell.

6. A method for a cultivation of microtissue, comprising:
(a) providing a membrane;
(b) forming a hole on the membrane;
(c) attaching said membrane to a carrier;
(d) causing the hole to form a microwell with the carrier;
(e) disposing plural cells on the membrane; and
(f) cultivating the plural cells to form a microtissue in the microwell.

7. An apparatus for microtissue cultivation, comprising:
a carrier; and
a membrane disposed on the carrier and having a hole where plural cells are cultivated to form a microtissue.

8. The apparatus according to claim 7, further comprising a substrate to cultivate the microtissue and to be cohered on a transplantation area to transplant the microtissue.

9. The apparatus according to claim 7, wherein the membrane is a hydrophobic membrane and the material of the membrane comprises at least one being selected from a group consisting of a siloxane, an alkene and a polycarbonate, and the carrier comprises at least one being selected from a group consisting of a glass, a ceramic, a polystyrene, a silicon wafer, a gelatin and a metal.
